(19) United States
(12) Patent Application Publication
(10) Pub. No.: US 2018/0264074 A1
(43) Pub. Date: Sep. 20, 2018

(54) USE OF POLYMYXIN AS AN ANTIDOTE FOR INTOXICATIONS BY AMATOXINS

(71) Applicant: UNIVERSIDADE DO PORTO, Porto (PT)

(72) Inventors: Felix CARVALHO, Porto (PT); Juliana GARCIA, Porto (PT); Maria DE LOURDES BASTOS, Porto (PT); Vera Marisa FREITAS COSTA, Porto (PT); Alexandra CARVALHO, Porto (PT); Ricardo SILVESTRE, Porto (PT); Jose Alberto RAMOS DUARTE, Porto (PT); Ricardo Jorge DINIS OLIVEIRA, Porto (PT)

(21) Appl. No.: 15/571,213

(22) PCT Filed: May 18, 2016
(86) PCT No.: PCT/IB2016/052905

§ 371 Date: Nov. 1, 2017

(30) Foreign Application Priority Data
May 18, 2015 (PT) 108481

(51) Int. Cl. A61K 38/12; A61P 39/02

(52) U.S. Cl. CPC A61K 38/12; A61P 39/02

(57) ABSTRACT

The present application refers to the development of a new effective antidote for the poisonous Amanita mushroom species, after exploring an innovative in silico and in vivo approach based on the binding site of amatoxin to RNA polymerase II (RNAPII) and the screening of clinical drugs with bioisosterism with amatoxins in the same models. Proof of concept was attained in vivo, using CD-1 mice, and clinical application is immediately proposed, in addition to the already prescribed therapeutic measures, taking advantage of well-established clinical use of the drug found to be an effective antidote, polymyxin B. Thus, Polymyxin and/or its derivatives/precursors consist in a therapeutic strategy on Amanita Phalloides as demonstrated by data gathered and showed in the present application.

### TECHNICAL FIELD

The present application refers to the development of a new effective antidote for the poisonous Amanita mushroom species.

### BACKGROUND

Amanita phalloides species are recognized to be involved in the majority of human deaths from mushroom poisoning. This species is widely distributed across Europe and Northern America and represent a global public health risk. It is difficult to estimate the exact number of poisoning cases that occur each year due to under-reporting procedures at hospital emergencies, but clinical records of patients admitted into ten Portuguese hospitals, between 1990 and 2008, showed 93 cases of acute poisoning by mushrooms. Of those, the hepatotoxic profile presentation occurred in 63.4%. The mortality in cases of hepatotoxicity was 11.8%. According to American statistics in 2012, a total of 6600 mushroom intoxications were reported to the National Poison Data System of the American Association of Poison Control Centers (AAPCC). Among these cases, 82.7% were attributed to unknown mushroom type, while cyclopeptides-containing mushrooms represented 44 cases (of those 4 patients died). The prominent toxic constituents of Amanita phalloides have been identified as cyclic octapeptides named amatoxins, mainly α-, β-, and γ-amatoxins. From those, α-amanitin accounts for about 40% of the amatoxins and is considered the main responsible for Amanita phalloides induced mortality and morbidity. Amatoxins bind and inhibit RNA polymerase II (RNAP II). This action mechanism results in the inhibition of transcription of mRNA and protein synthesis, causing mainly liver and kidney necrosis. Most patients die within a few days unless organ transplant occurs quickly. Unfortunately, so far, no good antidote for mushroom poisonings was found. The used treatments, namely antibiotics (benzylpenicillin, ceftazidime), N-acetylcystein, and silybin show poor therapeutic efficacy. The high lethality and the high cost per patient in the intensive care, mainly when organ transplant is required, makes this medical emergency a burden to families and health care providers and systems. In the present application is described a new use for polymyxin B and polymyxin derivatives/precursors as an antidote against amatoxin-containing mushrooms, based on in silico and in vivo studies already performed. For ethical reasons, polymyxin B should be added to the currently used and poorly effective emergency protocol used in each hospital. Polymyxin B has a well established use in hospital protocols for multiresistant bacteria, thus its safety is already guarantied.

### SUMMARY

The present application discloses a polypeptide for use as an antidote for amatoxins poisonings in mammals, wherein the polypeptide comprises binding proprieties on RNAP II at the residues Arg726, Ile 759, Ala759, Gln760 and/or Gln767.

In a further embodiment, the polypeptide does not comprise binding properties at TL and bridge helix residues of RNAP II.

In another embodiment, the polypeptide is Polymixin B.

In even another embodiment, the polypeptide is Polymixin B derivatives and/or Polymixin B precursors.

In another embodiment, the polypeptide is administrated in a therapeutically effective dose of 1.5-2.5 mg/kg/day in single or multiple doses.

### General Description

The present application refers to an effective antidote for intoxication with amatoxins-containing mushrooms in mammals. Therefore, in silico methodologies were applied to evaluate peptides with similar composition and molecular weight to that amatoxins for putative competition and displacement from its binding site in RNAP II. In silico results show that polymyxin B binds to RNAPII in the same interface of α-amanitin, showing this way potential to compete with this toxin without interfering with RNAPII activity, and hence protecting RNAP II from α-amanitin-induced inhibition (FIG. 1).

Following the in silico studies, in vivo studies were performed to prove the efficacy of polymyxin B in amatoxin poisoning. For this purpose, adult male mice (CD-1) were used. Two experiments were performed to test polymyxin B effectiveness: polymyxin B was administered concomitantly with α-amanitin and four hours after administration of α-amanitin. Concomitant therapy consisted of 0.33 mg/kg of α-amanitin followed by 2.5 mg/kg of polymyxin B (one administration). In the second experiment, three 2.5 mg/kg administrations of polymyxin B were used in different time-points [4, 8 and 12 h, intraperitoneal (i.p.) administration] after one α-amanitin (dose 0.33 mg/kg i.p.) exposure, as to mimic the clinical scenario of late intoxication diagnosis; human intoxication is often only found hours later when symptoms become clinically relevant. The results show that all animals exposed to the single dose of amanitin died until day 5, whereas 100% of animals concomitantly treated with polymyxin B survived until the 30th day of the experiment (FIG. 2), without major signs of injury or discomfort. Moreover 50% of animal exposed to polymyxin B 4, 8 and 12 h after α-amanitin survived (FIG. 2). In order to validate and unveil some mechanisms involved, an acute study with the same doses and scheme was performed, with animals being sacrificed 24 h after α-amanitin administration. Histological and plasma data showed that polymyxin B protected against hepatic and renal damage caused by α-amanitin (FIG. 3 and FIG. 4).

### BRIEF DESCRIPTION OF THE DRAWINGS

Without intent to limit the disclosure herein, this application presents attached drawings of illustrated embodiments for an easier understanding.

FIG. 1. Survival curves after concomitant i.p. administration of 0.33 mg/kg of α-amanitin and polymyxin B (Ama+Pol— 2.5 mg/kg) and 3 administrations of polymyxin B (Ama+Pol— 3×2.5 mg/kg) at different time-points (4, 8 and 12 h, i.p. administration) after one α-amanitin (dose 0.33 mg/kg i.p.). Results are expressed in percent survival. Control tests were performed (Control), consisting of a saline-control treatment. A polymyxin treatment (Pol) was additionally performed. A treatment with α-amanitin (Ama) as also made.

FIG. 2. Plasma levels of Aspartate aminotransferase (AST) and alanine aminotransferase (ALT) in control, polymyxin B (3×2.5 mg/kg) (Pol), α-amanitin (Ama) and α-amanitin+polymyxin B (3×2.5 mg/kg) (Ama+Pol) groups. Results are presented as means±standard deviation (SD), and were obtained from 4-5 animals from each treatment. Statistical comparisons were made using Kruskal-Wallis ANOVA on Ranks followed by the Dunn's post hoc test (*p<0.05, Ama vs. control; #p<0.05, Ama vs. Ama+Pol).

FIG. 3. Liver histopathology by light microscopy. (A) Light micrograph (40×) from α-amanitin treated-group. The presence of cellular oedema (1), cytoplasmic vacuolization (2), interstitial inflammatory cell infiltration (3), as well as some necrotic zones can be seen (4). (B) Light micrograph (40×) from α-amanitin+polymixin B (3×2.5 mg/kg) group. The oedema, cytoplasmatic vacuolization and necrosis, were significantly attenuated. Increase number interstitial inflammatory cell was still observed.

FIG. 4. Kidney histopathology by light microscopy (A) Light micrograph (10×) from α-amanitin-treated group. The presence of cytoplasmatic vacuolization (5), renal corpuscles with a wide capsular space, and thickened external Bowman capsule (6), as well as some necrotic zones can be seen (7). The presence large amounts of fibrin-related material cause enlargement and obstruction of distal tubules (8). (B) Light micrograph (10×) from α-amanitin+polymixin B (3×2.5 mg/kg) group. The oedema, cytoplasmatic vacuolization and necrosis, were significantly attenuated.

### DETAILED DESCRIPTION

For the development of an effective antidote for intoxication with amatoxin-containing mushrooms in humans, in silico methodologies were applied to evaluate peptide compounds with similar composition and molecular weight to that amatoxins for putative competition and displacement from its binding site in RNAP II. Docking and molecular dynamics (MD) simulation coupled with molecular mechanics-generalized born surface area method (MM-GBSA) energy decomposition were carried out to clarify the inhibition mechanism of RNAP II by α-amanitin and to provide a new insight into the plausible mechanism of action of three antidotes (benzylpenicillin, ceftazidime and silybin) used in amatoxin poisoning.

Results revealed that α-amanitin should affect RNAP II transcription by compromising trigger loop (TL) function. The observed direct interactions between α-amanitin and residues Leu1081, Asn1082 Thr1083 His1085 and Gly1088 alters the elongation process and thus contribute to the inhibition of RNAP II. We also present evidences that α-amanitin can interact directly with the bridge helix residues Gly819, Gly820 and Glu822, and indirectly with His816 and Phe815. This destabilizes the bridge helix, possibly causing RNAP II activity loss. These results clearly reinforces the hypothesis of an important role of the bridge helix and TL in the elongation process and are consistent with the existence of a network of functional interactions between the bridge helix and TL that control fundamental parameters of RNA synthesis.

Benzylpenicillin, ceftazidime and silybin are able to bind to the same site as α-amanitin, although not replicating the unique α-amanitin binding mode. They establish considerably less intermolecular interactions and the ones existing are essential confine to the bridge helix and adjacent residues. Therefore, the therapeutic effect of these antidotes does not seem to be directly related with binding to RNAP II.

RNAP II α-amanitin binding site can be divided into specific zones with different properties providing a reliable platform for the structure-based drug design of novel antidotes for α-amatoxin poisoning. An ideal drug candidate should be a competitive RNAP II binder that interacts with Arg726, Ile756, Ala759, Gln760 and Gln767, but not with TL and bridge helix residues. In silico results show that polymyxin B binding site is located in the same interface of α-amanitin, which can prevent the toxins from to binding, and hence protecting RNAP II from α-amanitin-induced impairment.

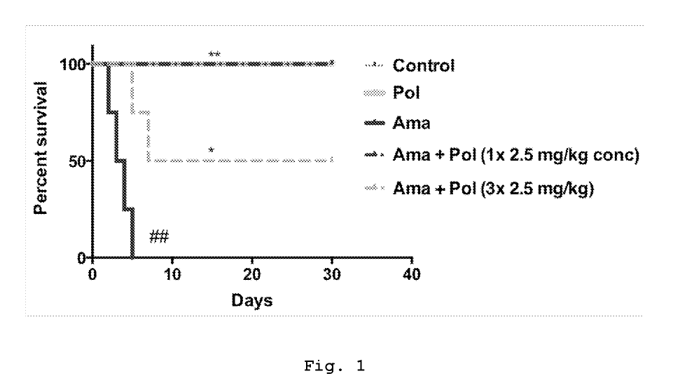

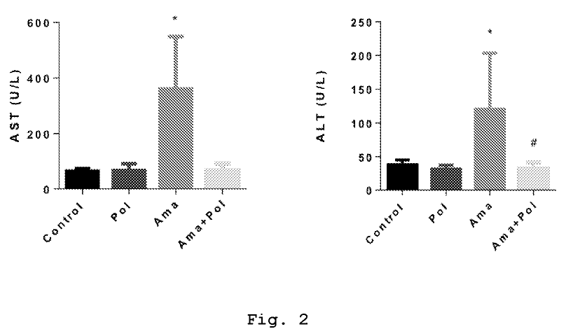

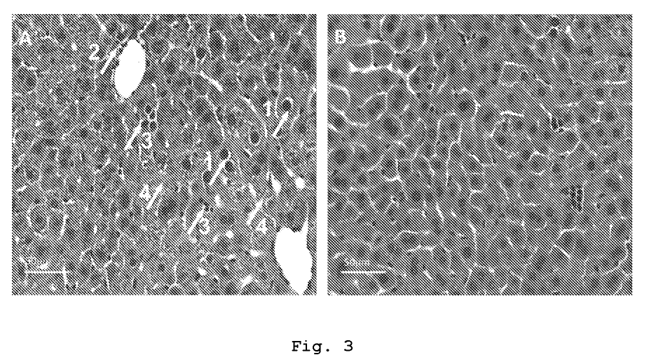

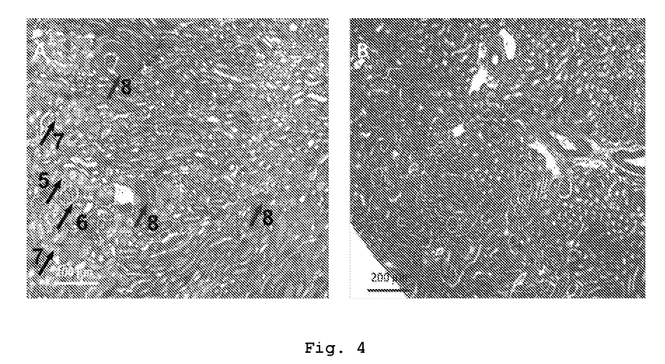

Following the in silico studies, in vivo studies were performed to prove the efficacy of polymyxin B in amatoxin poisoning. For this purpose, adult male mice (CD-1) were used. Two experiments were performed to test polymyxin B effectiveness: polymyxin B was administered concomitantly with α-amanitin and four hours after administration of α-amanitin. Concomitant therapy consisted of 0.33 mg/kg of α-amanitin followed by 2.5 mg/kg of polymyxin B (one administration). In the second experiment, three 2.5 mg/kg administrations of polymyxin B were used in different time-points (4, 8 and 12 h, i.p. administration) after one α-amanitin exposure (0.33 mg/kg i.p dose), as to mimic the clinical scenario of late intoxication diagnosis; human intoxication is often only found hours later when symptoms become clinically relevant. The results show that all animals exposed to the single dose of amanitin died until day 5, whereas 100% of animals treated with concomitant polymyxin B survived until the 30th day of the experiment (FIG. 1). Moreover, 50% of animals exposed to polymyxin B 4, 8 and 12 h after α-amanitin survived (FIG. 1). In order to validate and unveil some mechanisms involved, an acute study with the same doses and scheme was performed with polymyxin B 4, 8 and 12 h after α-amanitin administration. Animals were sacrificed 24 h after α-amanitin administration. Plasma biochemistry shows that plasma aminotransferases were increased in the α-amanitin-intoxicated group, while this effect was totally reverted with administration of multiple doses of 2.5 mg/kg polymyxin B (FIG. 2). Promising results were also demonstrated through histology. Histological analysis of the liver from the α-amanitin-intoxicated group showed the presence of cellular oedema, cytoplasmic vacuolization and interstitial inflammatory cell infiltration, as well as some necrotic zones (FIG. 3) On the other hand, the multiple administration of polymyxin B resulted in a significant reversion against α-amanitin-induced necrotic changes as well as the induced oedema and cytoplasmic vacuolization (FIG. 3). Histological examination of α-amanitin-intoxicated kidney revealed degenerative changes. The renal corpuscles appearance is heterogeneous, with a wide capsular space, and thickened external Bowman capsule. Proximal tubules showed histological changes in the form of necrotic cells, vacuolation and oedema (FIG. 4). Marked atrophy and degeneration of distal tubules cells was also observed, and large amounts of fibrin-related material caused enlargement and obstruction of these tubules. Noteworthy, the administration of polymyxin B protected against the occurrence of the above referred alterations, particularly the necrosis and the obstruction of distal tubules (FIG. 4).

In silico or in vivo studies demonstrated that polymyxin B acts on RNAP II, preventing α-amanitin binding. Clinical assays in intoxicated humans are feasible with polymyxin B, as according to the allometric scalling standardly used the 3 doses of 2.5 mg/kg of polymyxin B in mice, sums up to approximately 1 mg/kg in humans, when the current recommended dose of iv polymyxin B for patients with normal renal function is 1.5-2.5 mg/kg/day in two divided doses administered as a 1 h infusion (Zavascki A P et al. 2007). Other dosing and therapeutic schemes are used presently in treatment of multidrug-resistant pathogens with injectable polymyxin B, but initial dose on intoxicated patients should follow the hospitals protocol for polymyxin B. For ethical reasons and as Amanita Phalloides ingestion has a high lethality, polymyxin B should be added to the present protocol on Amanita Phalloides intoxication as to improve the overall survival of patients.

1. A polypeptide for use as an antidote for amatoxins poisonings in mammals, wherein the polypeptide comprises binding proprieties on RNAP II at the residues Arg726, Ile 759, Ala759, Gln760 and/or Gln767.

2. The polypeptide for the use according to claim 1, wherein said polypeptide does not comprise binding properties at TL and bridge helix residues of RNAP II.

3. The polypeptide for the use according to claim 1, wherein said polypeptide is Polymixin B.

4. The polypeptide for the use according to claim 3, wherein said polypeptide is Polymixin B derivatives and/or Polymixin B precursors.

5. A method for treating amatoxins poisoning in mammals comprising administering the polypeptide of claim 1, wherein said polypeptide is administrated in a therapeutically effective dose of 1.5-2.5 mg/kg/day in single or multiple doses to the mammal in need thereof.

6. An antidote for amatoxins poisoning in mammal comprising the polypeptide according to claim 1.
